(19) United States
(12) Patent Application Publication
(10) Pub. No.: US 2013/0096464 A1
(43) Pub. Date: Apr. 18, 2013

(54) SOUND PROCESSING APPARATUS AND BREATHING DETECTION METHOD

(71) Applicant: FUJITSU LIMITED, Kawasaki-shi (JP)

(72) Inventors: Masakiyo TANAKA, Kawasaki-shi (JP); Masanao Suzuki, Yokohama (JP)

(73) Assignee: FUJITSU LIMITED, Kawasaki-shi (JP)

(21) Appl. No.: 13/693,711

(22) Filed: Dec. 4, 2012

Related U.S. Application Data
(63) Continuation of application No. PCT/JP2010/059877, filed on Jun. 10, 2010.

(51) Int. Cl. A61B 7/00

(52) U.S. Cl. CPC A61B 7/003
USPC 600/586

(57) ABSTRACT

A sound processing apparatus comprising a processor configured to convert an input audio signal into a frequency domain signal; calculate similarity of the current frequency domain signal and a previous frequency domain signal; and determine a breathing state of a biological entity indicated by the audio signal, based on the similarity.

### CROSS REFERENCE TO RELATED APPLICATIONS

This application is a continuation application of International Application PCT/JP2010/059877, filed on Jun. 10, 2010 and designating the U.S., the entire contents of which are incorporated herein by reference.

### FIELD

The embodiments discussed herein are related to a sound processing apparatus and a breathing detection method.

### BACKGROUND

Conventionally, with respect to technology that detects the state of breathing during sleep, one approach has been to detect the state of breathing by detecting breath sounds, dividing the detected breath sounds into plural frequency blocks by a band-pass filter, etc., and comparing a detection value (voltage value) of each divided block with a predetermined threshold (see, e.g., Japanese Laid-Open Patent Publication No. 2007-289660).

Since breathing differs among individuals and even changes in various ways with the same individual depending on sleeping conditions, such as changes in the method of breathing (e.g., switching from nose breathing to mouth breathing) and changes in sleeping position (e.g., switching from sleeping on one's side to sleeping face down or on one's back), the frequency characteristics of breathing change. Therefore, according to the conventional technology, the method of using a threshold for breathing detects no breathing for a detected value that is below the threshold. If the detected value of noise exceeds the threshold, this method detects the noise as breathing. Thus, the conventional technology has a problem of erroneously determining the state of breathing when there are differences among individuals or a change in the state of breathing.

### SUMMARY

According to an aspect of an embodiment, a sound processing apparatus includes a processor configured to convert an input audio signal into a frequency domain signal; calculate similarity of the current frequency domain signal and a previous frequency domain signal; and determine a breathing state of a biological entity indicated by the audio signal, based on the similarity.

The object and advantages of the invention will be realized and attained by means of the elements and combinations particularly pointed out in the claims.

It is to be understood that both the foregoing general description and the following detailed description are exemplary and explanatory and are not restrictive of the invention.

### BRIEF DESCRIPTION OF DRAWINGS

FIG. 1 is a block diagram of a schematic configuration of a sound processing apparatus;

FIG. 2 depicts one example of a breathing state during sleep;

FIG. 3 depicts frequency characteristics of a single breath;

FIG. 4 is a block diagram of the sound processing apparatus of a first embodiment;

FIG. 5 depicts distribution of a signal to be input to a similarity calculating unit;

FIG. 6 is a diagram for explaining a similarity calculation at the similarity calculating unit;

FIG. 7 is a flowchart of processing performed by the similarity calculating unit;

FIG. 8 depicts an example of a similarity plot for a calculation of a duration period by a duration calculating unit;

FIG. 9 is a diagram of a processing example of a duration period calculation by the duration calculating unit;

FIG. 10 is a flowchart of overall processing of breathing detection according to the first embodiment;

FIG. 11 is a block diagram of the sound processing apparatus according to a second embodiment;

FIG. 12 is a diagram for explaining background noise removal at the similarity calculating unit;

FIG. 13 is a flowchart of background noise removal processing performed by the similarity calculating unit;

FIG. 14 is a flowchart of the similarity calculating processing according to a third embodiment;

FIG. 15 is a flowchart of the similarity calculating processing according to a fourth embodiment;

FIG. 16 is a flowchart of the similarity calculating processing according to a fifth embodiment;

FIG. 17 is a block diagram of the sound processing apparatus according to a sixth embodiment;

FIG. 18 is a diagram for describing a non-breathing state; and

FIG. 19 is a flowchart of apnea determining processing of the sixth embodiment.

### DESCRIPTION OF EMBODIMENTS

Preferred embodiments of a sound processing apparatus and a breathing detection method will be described in detail with reference to the accompanying drawings. The sound processing apparatus and the breathing detection method accurately determine the breathing state, using breathing cycles during sleep, the duration of one breath, and similarity of frequency characteristics of temporally proximal breaths.

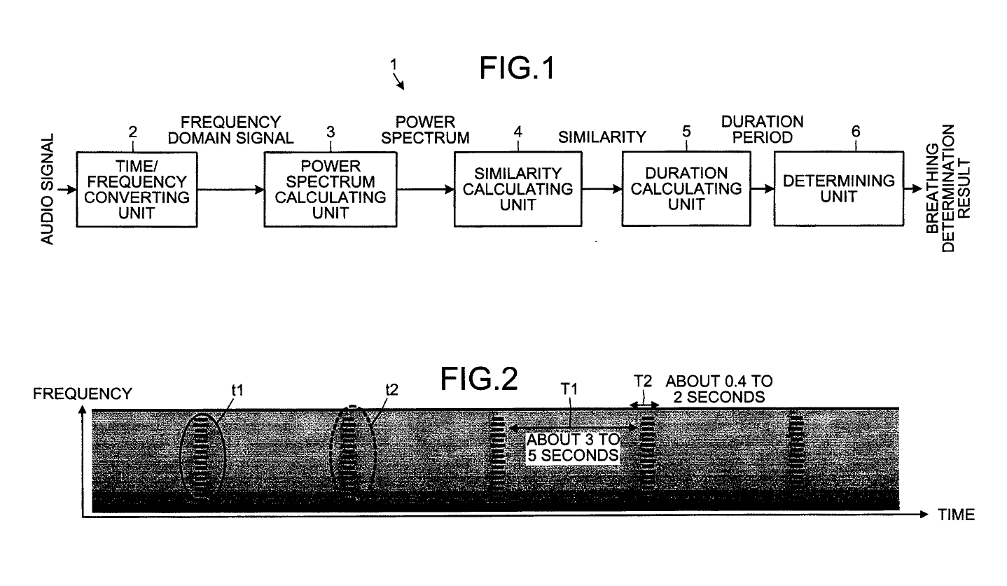

FIG. 1 is a block diagram of a schematic configuration of the sound processing apparatus. The sound processing apparatus detects the presence or absence of breathing, based on sounds by a human being (biological entity) during sleep. This sound processing apparatus 1 has a time/frequency converting unit 2, a power spectrum calculating unit 3, a similarity calculating unit 4, a duration calculating unit 5, and a determining unit 6.

The time/frequency converting unit 2 receives an input of a digital audio signal divided into frames according to a given sampling rate and converts this temporally varying audio signal into a frequency domain signal. The power spectrum calculating unit 3 calculates a temporal power spectrum of the frequency domain signal resulting from the conversion of the audio signal by the time/frequency converting unit 2. The similarity calculating unit 4 calculates the similarity of the current power spectrum calculated by the power spectrum calculating unit 3 and a past power spectrum of a predetermined range. The duration calculating unit 5 calculates the duration of the similarity calculated by the similarity calculating unit 4. The determining unit 6 determines the presence or absence of breathing based on the duration calculated by the duration calculating unit 5.

FIG. 2 depicts one example of a breathing state during sleep. The horizontal axis represents time and the vertical axis represents frequency. As depicted in the example, human breathing includes a breathing cycle T1 and one-breath duration period T2. The breathing cycle T1 is on the order of 3 to 5 seconds and the one-breath duration period T2 is on the order of 0.4 to 2 seconds. As depicted, during sleep, the one-breath duration period T2 is continuously repeated at the breathing cycle T1.

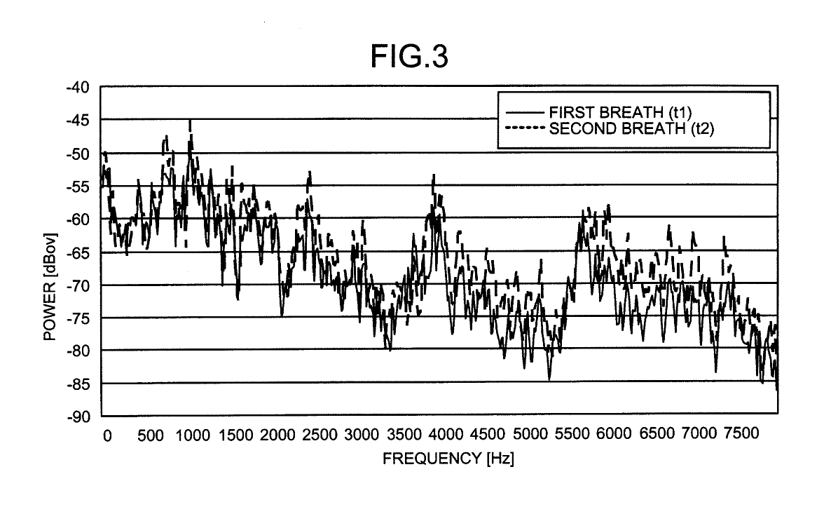

FIG. 3 depicts frequency characteristics of a single breath. The horizontal axis represents frequency and the vertical axis represents electrical power. The frequency characteristics are shown for the 2 adjacent breaths (time t1 and time t2) depicted in FIG. 2. Thus, the frequency and power characteristics for the first breath during time t1 are similar to those for the second breath during time t2.

The sound processing apparatus accurately determines the breathing state by performing processing utilizing the above properties of breathing states during sleep. Breathing (the person is breathing) is determined when a signal having frequency characteristics similar to those of the current and past signals that include the breathing cycle T1 is continuously present for a given period of time (the duration period T2). Thus, the sound processing apparatus determines the current signal, which continues to be similar to the past signals for a given period of time, to indicate breathing and therefore, can correctly detect breathing even when the breathing is indicated by minimal electrical power (breath sounds during sleep are small) and exclude noise indicated by significant electrical power, thereby preventing erroneous detection. Consequently, the presence or absence of breathing can be accurately determined irrespective of the magnitude of the electrical power and surrounding environmental conditions.

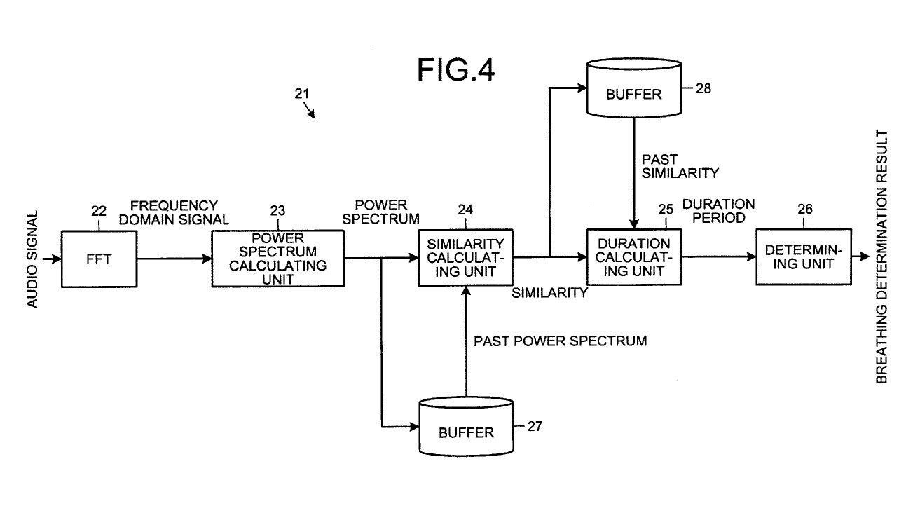

FIG. 4 is a block diagram of the sound processing apparatus of a first embodiment. A sound processing apparatus 21 of the first embodiment is of the configuration depicted in FIG. 1. The time/frequency converting unit 2 includes an FFT 22 and converts, by the fast Fourier transform, an input signal (audio signal) to a frequency domain signal. The time/frequency converting unit 2 may use another time/frequency converting unit without using the FFT 22. The power spectrum calculating unit 23 calculates a square sum of a real part and an imaginary part of each band of the frequency domain signal and calculates the power spectrum. Past power spectrum data for a predetermined period and calculated by the power spectrum calculating unit 23 is stored in a buffer 27.

The similarity calculating unit 24 compares the current power spectrum and a past power spectrum stored in the buffer 27 to calculate the similarity. Past similarity data for a predetermined period and calculated by the similarity calculating unit 24 is stored in a buffer 28. The duration calculating unit 25 compares the current similarity with past similarity stored in the buffer 28 to calculate the duration period. The determining unit 26 determines the state as a “breathing state” if the duration period calculated by the duration calculating unit 25 is within a predetermined range (T2).

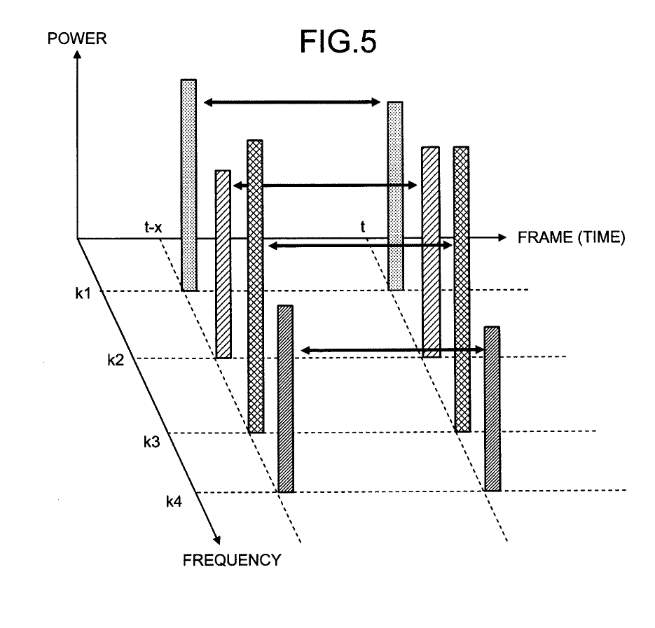

FIG. 5 depicts distribution of the input signal to be input to the similarity calculating unit. Two horizontal axes represent frame (time) and frequency, respectively, and the vertical axis represents electrical power. The similarity calculating unit 24 compares a current (time t) frame and a previous frame of the same frequency band, i.e., compares the frames of k1, compares the frames of K2, compares the frames of k3, and compares the frames of k4 in FIG. 5. A comparison range for making comparison with a previous frame is a period equal to 1 breathing cycle T1 and is a range of x (x1≦x≦x2) before t. In the above example, x1 and x2 are x1=3 and x2=5. At frequency k1 in FIG. 5, the power spectra of the frames at time t and time (t−x) are compared. Likewise, at frequencies k2 to k4, the power spectra are compared at each frequency band. The similarity calculating unit 24 integrates the comparison results at individual frequency bands into one and calculates the similarity of the frame t and the frame (t−x).

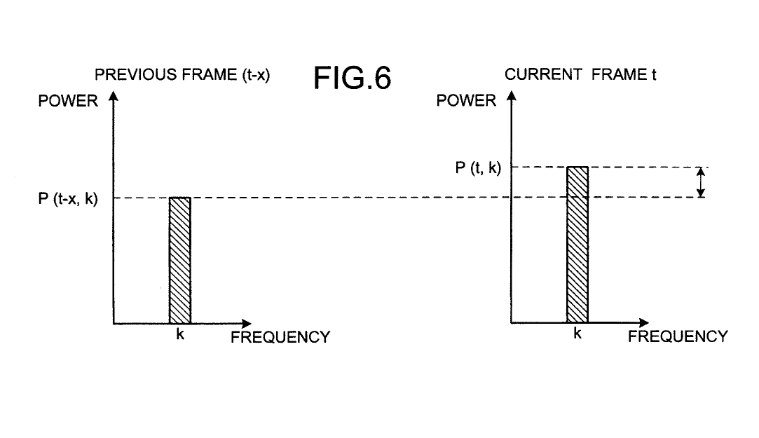

FIG. 6 is a diagram for explaining the similarity calculation at the similarity calculating unit. The horizontal axis represents frequency and the vertical axis represents electrical power. The similarity calculating unit 24 calculates for each frequency band, the difference of the power spectra of the current frame t and the previous frame (t−x) at a range of x (x1≦x≦x2) from the current frame. The comparison of the previous frame (t−x) and the current frame t at one frequency band k will now be described with reference to FIG. 6. The similarity calculating unit 24 sets a predetermined threshold TH, using the electrical power of the previous frame (t−x) as a reference. The threshold TH can be, for example, on the order of 3 dB.

The similarity is determined using the following equation:

|P(t,k)−P(t−x,k)|≦TH

If the above equation is satisfied, the flag is flag(x,k)=1. Namely, when the electrical power of the current frame t relative to the electrical power of the previous frame (t−x) is less than or equal to threshold TH, it is determined that “there is a similarity” and the flag is set to “1”. Conversely, when the electrical power of the current frame t relative to the electrical power of the previous frame (t−x) is greater than threshold TH, it is determined that “there is no similarity” and the flag is set to “0”.

The similarity calculating unit 24 performs the above processing with respect to all frequency bands and regards the sum of the flags of all frequency bands as shown in the following equation as similarity.

$$similarity ( t , x ) = {\sum }_{k = 1}^{K} flag ( x , k )$$

Thereafter, the similarity calculating unit 24 calculates the similarity with respect to each value of x satisfying x1≦x≦x2 (i.e., 1 breathing cycle T1).

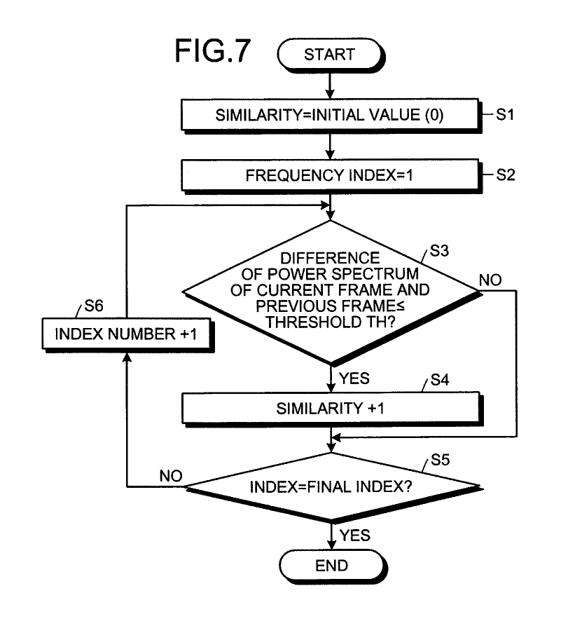

FIG. 7 is a flowchart of processing performed by the similarity calculating unit. As depicted in FIG. 7, the similarity calculating unit 24 first sets the similarity to the initial value (0) (step S1). The similarity calculating unit 24 then sets the index of frequency k as 1 (index=1) (step S2). The similarity calculating unit 24 compares the power spectrum of the current frame with the power spectrum of the previous frame and determines if the difference is less than or equal to threshold TH (step S3). If the electrical power of the power spectrum of the current frame is less than or equal to threshold TH based on the electrical power of the power spectrum of the previous frame (step S3: YES), the similarity calculating unit 24 adds 1 to the similarity (step S4).

On the other hand, if the electrical power of the power spectrum of the current frame is greater than threshold TH based on the electrical power of the power spectrum of the previous frame (step S3: NO), the similarity calculating unit 24 goes to step S5 without adding 1 to the similarity. At step S5, the similarity calculating unit 24 determines if the index is the final index and if not (step S5: NO), the similarity calculating unit 24 shifts the frequency k (index number+1) (step S6) and returns to step S3. On the other hand, if the index is the final index (step S5: YES), the similarity determination has been completed with respect to all frequencies and the processing is finished.

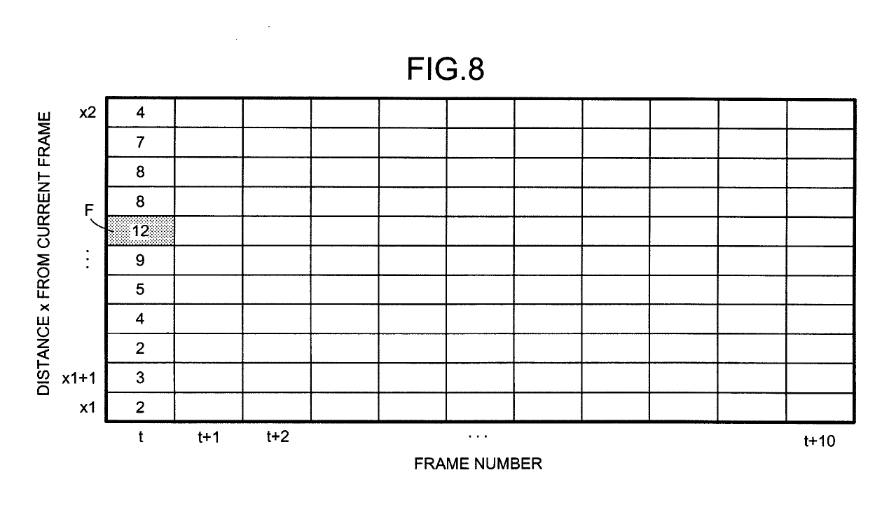

FIG. 8 depicts an example of a similarity plot for the calculation of the duration period by the duration calculating unit. The horizontal axis represents frame number and vertical axis represents distance x from the current frame. The similarity calculating unit 24 plots (identifies) a similarity (value thereof) greater than a predetermined threshold at a corresponding area of a matrix storage field. For example, the example depicted in FIG. 8 represents a state in which the similarity of frame t has been plotted and indicates a value of the similarity, for the sake of convenience, by a numerical value in each area for each distance x from the current frame.

In terms of the value of the similarity, the duration calculating unit 25 determines a similarity exceeding the threshold as a high similarity. For example, if the threshold is set at 10, the hatched area of the similarity value “12” exceeding the threshold 10 is affixed with an identifier F and is stored to the buffer 28. Therefore, in practice, the values depicted in FIG. 8 are not stored and a similarity greater than the threshold is plotted using the identifier F. A value corresponding to the number of bands of frequency k in the power spectrum calculating unit 23 is set as this threshold and a value of 20 to 30% of the number of bands is set as the threshold.

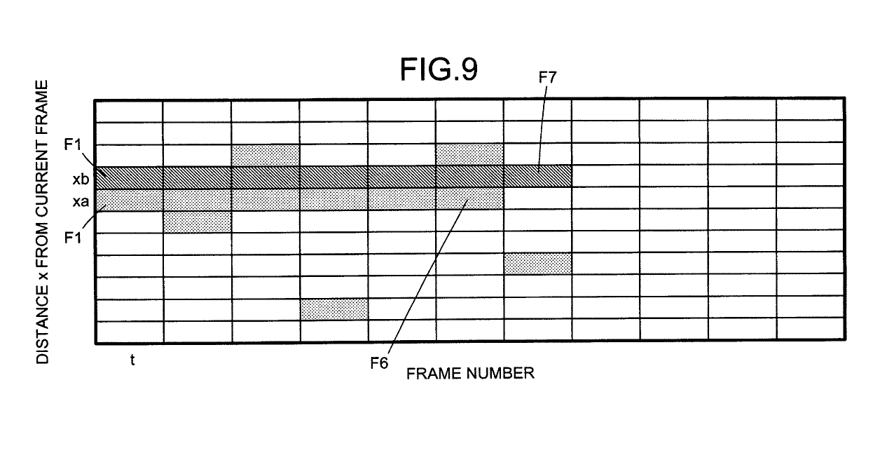

FIG. 9 is a diagram of a processing example of the duration period calculation by the duration calculating unit. If identifier F of a similarity exceeding the threshold depicted in FIG. 8 is plotted consecutively, for example, the plot results as depicted in FIG. 9. When plural identifiers F are affixed to frames at the same distance x but have different frame numbers, the duration calculating unit 25 detects the number of such continuous frames and outputs the number as a corresponding duration period. In the example depicted in FIG. 9, at distance xa, identifier F continues for six frames (F1 to F6) and at distance xb, identifier F continues for seven frames (F1 to F7).

The continued frames at distance xa and distance xb are determined to have finished the frame continuation when the value of the similarity becomes lower than the threshold. In FIG. 9, at distance xa, the similarity becomes lower than the threshold and the continuation is finished after six consecutive frames but at distance xb, the similarity becomes lower than the threshold at seven consecutive frames. Basically, the duration period is obtained using the greatest number of continued frames but when multiple distances x have the number of the continued frames, the duration period is obtained by performing the following processing:

(1) When a start frame of the duration period in which the similarity is greater than or equal to the threshold at distance xb is the same frame as the start frame of a duration period in which the similarity is greater than or equal to the threshold at distance xa or a frame previous thereto, the duration period at distance xa is not used and the duration period corresponding to the continued frames at distance xb is obtained.
(2) In cases other than (1) above, the duration period corresponding to the continued frames at distance xa is obtained.

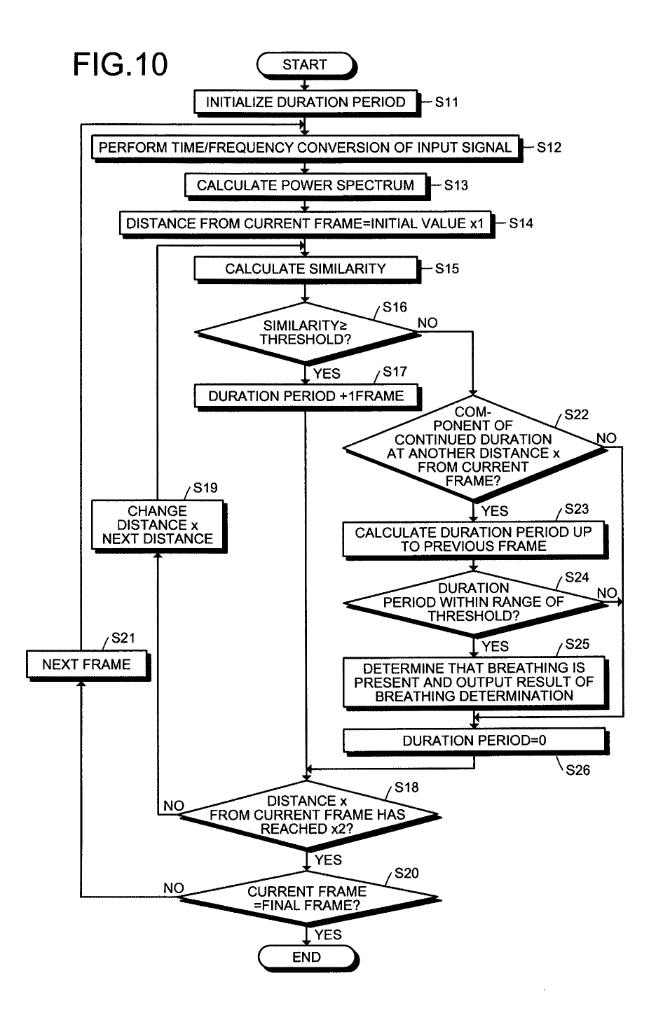

FIG. 10 is a flowchart of overall processing of the breathing detection according to the first embodiment. First, the duration calculating unit 25 initializes (resets) the duration period (step S11). The FFT 22 performs the time/frequency conversion of the input signal (step S12). The power spectrum calculating unit 23 calculates the temporal power spectrum of the frequency domain signal (step S13). The duration calculating unit 25 sets the distance from the current frame to the initial value x1 (three seconds in the above example) (step S14).

The similarity calculating unit 24 calculates the similarity by the processing depicted in FIG. 7 (step S15). The duration calculating unit 25 determines if the similarity is greater than or equal to the threshold (step S16). If the similarity is greater than or equal to the threshold (step S16: YES), the duration calculating unit 25 affixes the above identification to the corresponding frame, adds one frame to the duration period (step S17), and determines if distance x from the current frame has reached x2 (five seconds in the above example) at same frequency k (step S18). If distance x from the current frame is less than x2 (step S18: NO), the duration calculating unit 25 changes distance x to the next distance (step S19) and continues to perform the similarity calculating processing (step S15) and subsequent processing with respect to the resulting distance x2.

At step S16, if the similarity of the input signal (frame) becomes less than the threshold (step S16: NO), the duration calculating unit 25 determines if there is a component (frequency) of continued duration at another distance x from the current frame (step S22). If there is a component of continued duration at another distance x from the current frame (step S22: YES), the duration calculating unit 25 calculates the duration period up to the previous frame (step S23).

The determining unit 26 determines if the duration period is within the range of the one-breath duration period T2 (y1≦y≦y2) (step S24). If the duration period is within the range of one-breath duration period T2 (step S24: YES), the determining unit 26 determines that breathing is present and outputs results of the breathing determination (step S25). The determining unit 26 resets the duration period to 0 (step S26) and the flow returns to step S18. On the other hand, at step S22, if there is no component of continued duration at another distance x from the current frame (step S22: NO) or at step S24, if the duration period is not within the range of one-breath duration period T2 (step S24: NO), the flow goes to step S26 where the duration period is reset to 0.

At step S18, if distance x from the current frame has reached x2 (five seconds in the above example) (step S18: YES), the duration calculating unit 25 determines if the current frame is the final frame (step S20) and if not (step S20: NO), the flow returns to step S12 to execute processing of the next frame (step S21). On the other hand, at step S20, if the current frame is the final frame (step S20: YES), the duration calculating unit 25 ends the breathing determining processing.

According to the first embodiment, breathing sound similarity is obtained by frequency band and if a similar signal has a given duration period, then it is determined that there is breathing. Therefore, even small breaths during sleep can be detected correctly and the presence or absence of the breathing can be determined accurately.

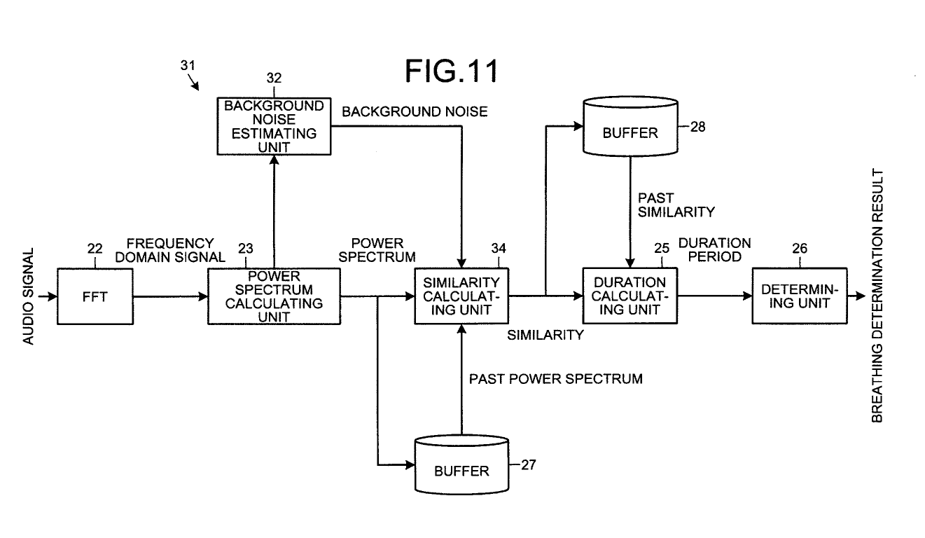

A second embodiment represents the first embodiment with an additional function of removing the background noise. FIG. 11 is a block diagram of the sound processing apparatus according to the second embodiment. Components identical to those described in FIG. 4 are given the same reference numerals used in FIG. 4. As depicted in FIG. 11, this sound processing apparatus 31 further includes a background noise estimating unit 32. The background noise estimating unit 32 estimates the magnitude of the background noise based on the power spectrum calculated by the power spectrum calculating unit 23. Namely, in the similarity determination, the background noise estimating unit 32 prevents an erroneous determination that breathing is present based only on the background noise despite the fact that no breathing is present, when the similarity becomes high in the same frequency band in which only the background noise is present.

In the estimation of the background noise, for example, in each frequency band, the value of the previous power is updated with the value of the current power when the power of the current frame is less than or equal to N times (e.g., twice) the power indicative of the estimated noise level at the previous frame. For example, the background noise pow(t,k)=COEFF×noise_pow (t−x, k)+(1−COEFF)×P(t,k) (P(t,k)≦2×noise_pow(t−x,k)) noise_pow(t−x,k) (otherwise) (where, COEFF is a constant.)

The above background noise estimating method is one example and processing may be performed of averaging the electrical power within a given period. Various processing can be used.

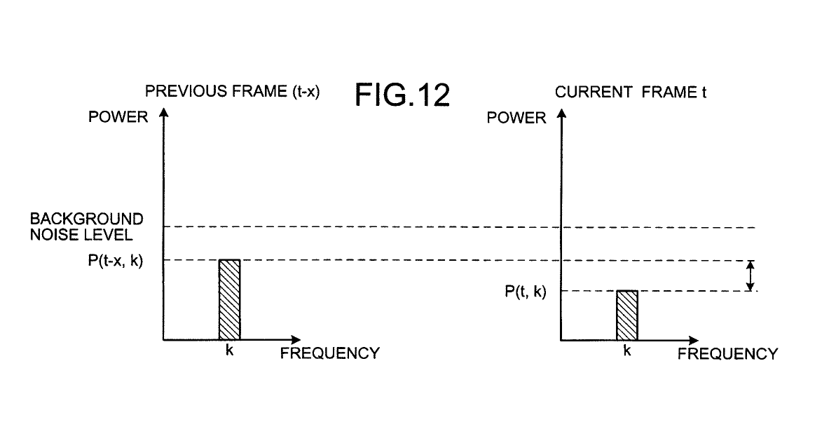

FIG. 12 is a diagram for explaining background noise removal at the similarity calculating unit. The similarity calculating unit 34, in the same manner as in the first embodiment, calculates for each frequency band, the difference of the power spectra of the current frame t and the previous frame (t−x) at a range of x (x1≦x≦x2) from the current frame. In the frequency band in which the electrical power is equal to or lower than the background noise level, however, the flag is set to “0”.

|P(t,k)−P(t−x,k)|≦TH

Namely, when the above conditions of the similarity determination are satisfied but the electrical power P(t,k) is of a level lower than the background noise level depicted in FIG. 12, flag(x,k)=0.

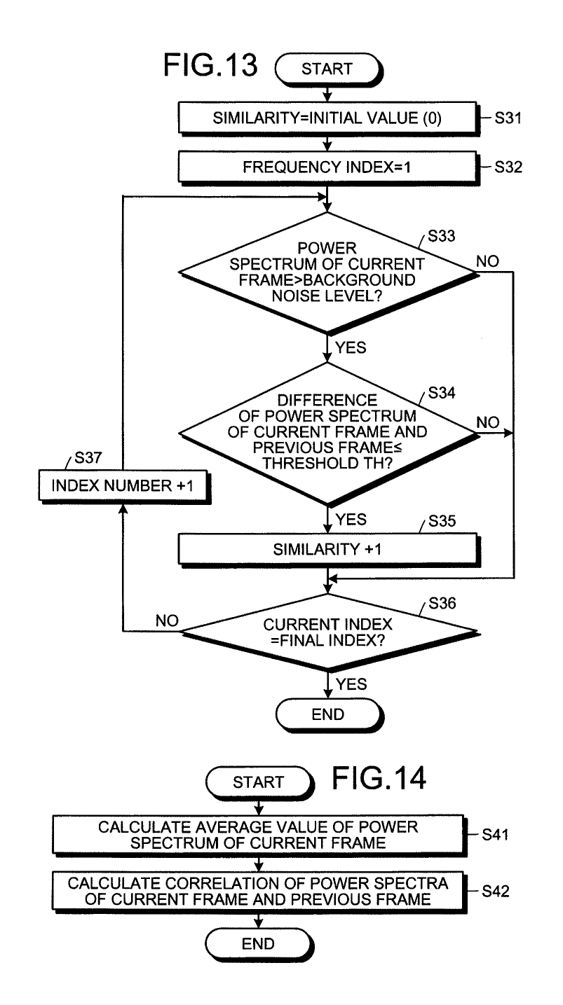

FIG. 13 is a flowchart of background noise removal processing performed by the similarity calculating unit. The similarity calculating unit 34 first sets the similarity to the initial value (0) (step S31). The similarity calculating unit 34 then sets the index of frequency k to 1 (index=1) (step S32). The similarity calculating unit 34 determines if the power spectrum of the current frame is greater than the background noise level (step S33). If the power spectrum of the current frame is greater than the background noise level (step S33: YES), the similarity calculating unit 34 continues to perform the processing at step S34 and thereafter; and if the power spectrum of the current frame is no greater than the background noise level (step S33: NO), the similarity calculating unit 34 goes to step S36 without performing the similarity adding processing, etc.

At step 33, if the power spectrum of the current frame is greater than the background noise level (step S33: YES), the similarity calculating unit 34 compares the power spectrum of the current frame with the power spectrum of the previous frame and determines if the difference is less than or equal to threshold TH (step S34). If the electrical power of the power spectrum of the current frame is less than or equal to threshold TH based on the electrical power of the power spectrum of the previous frame (step S34: YES), the similarity calculating unit 34 adds 1 to the similarity (step S35). On the other hand, if the electrical power of the power spectrum of the current frame is greater than threshold TH based on the electrical power of the power spectrum of the previous frame (step S34: NO), the similarity calculating unit 34 goes to step S36 without performing the addition of the similarity. At step S36, the similarity calculating unit 34 determines if the current index is the final index and if not (step S36: NO), the similarity calculating unit 34 shifts frequency k (index number +1) (step S37) and returns to step S33. On the other hand, if the current index is the final index (step S36: YES), the similarity determination has been completed with respect to all frequencies and the processing is finished.

According to the second embodiment, similarity can be prevented from being heightened between frames in which only the background noise is present and the breathing state can be detected more accurately.

In a third embodiment, another configuration of the similarity calculating unit will be described. The third embodiment uses a correlation of the power spectra calculated by the power spectrum calculating unit 23 as the similarity. The similarity calculating unit of the third embodiment uses the components depicted in the first embodiment but internal processing of the similarity calculating unit 24 differs. While various methods are conceivable for the correlation calculation, a general correlation equation using a correlation coefficient such as the following equation can be used.

$$\begin{matrix}similarity ( t , x ) = cor ( t , x ) \\ = \frac{{\sum }_{k = 1}^{K} ( P ( t , k ) - \overline{P ( t )} ) ( P ( t - x , k ) - \overline{P ( t - x )} )}{\sqrt{{\sum }_{k = 1}^{K} {( P ( t , k ) - \overline{P ( t )} )}^{2}} \sqrt{{\sum }_{k = 1}^{K} {( P ( t - x , k ) - \overline{P ( t - x )} )}^{2}}}\end{matrix}$$

P̅(̅t̅)̅: average value of power spectrum of frame t

FIG. 14 is a flowchart of the similarity calculating processing according to the third embodiment. The similarity calculating unit 24 of the third embodiment calculates, with respect to the power spectrum calculated by the power spectrum calculating unit 23, an average value of the power spectrum of the current frame t (step S41) and then calculates the correlation of the power spectra of the current frame and the previous frame, using the above correlation equation (step S42). The duration calculating unit 25 at the subsequent step calculates the duration period of the frame, using the output correlation value. According to the third embodiment, similarity can be calculated using a general-purpose correlation equation.

A fourth embodiment is configured to prevent an erroneous determination consequent to background noise as described by the second embodiment and uses power spectra correlation as similarity as described by the third embodiment. The fourth embodiment uses the components depicted in the second embodiment but the internal processing of the similarity calculating unit 34 differs. With respect to the calculation of the correlation, for example, the general correlation equation described in the third embodiment can be used. In the fourth embodiment as well, in the same manner as in the third embodiment, description will be made by the example of calculating the correlation using the average value of the power spectrum of the frame.

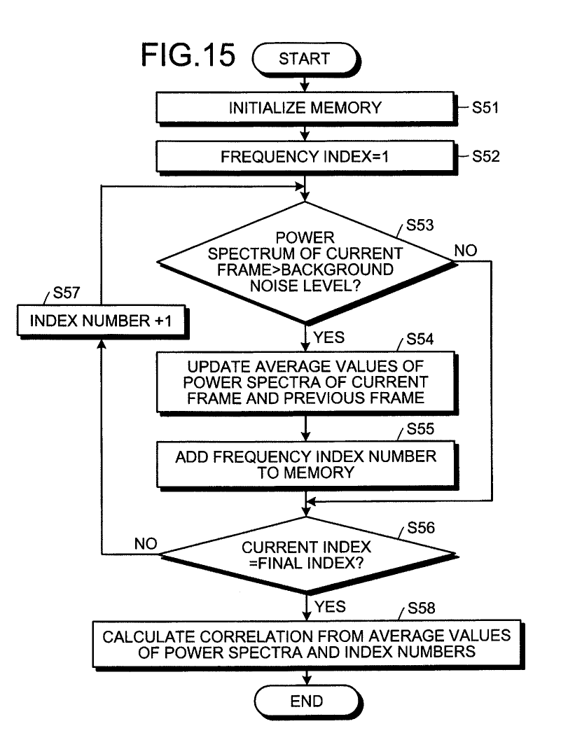

FIG. 15 is a flowchart of the similarity calculating processing according to the fourth embodiment. The similarity calculating unit 34 first initializes the memory (step S51). The memory to be initialized is a memory to store the average value of the power spectrum of the current frame and the average value of the power spectrum of the previous frame (buffer 27 depicted in FIG. 11). The memory disposed in the background noise estimating unit 32 to hold the index of the frequency band in which the power spectrum is greater than or equal to the background noise level is also included among the memory to be initialized.

The similarity calculating unit 34 sets the index of frequency k to 1 (index=1) (step S52). The similarity calculating unit 34 determines if the power spectrum of the current frame is greater than the background noise level (step S53). If the power spectrum of the current frame is greater than the background noise level (step S53: YES), the similarity calculating unit 34 continues to perform the processing of step S54 and thereafter, but if the power spectrum of the current frame is not greater than the background noise level (step S53: NO), the flow goes to step S56.

At step S53, if the power spectrum of the current frame is greater than the background noise level (step S53: YES), the similarity calculating unit 34 updates the average values of the power spectra of the current frame and the previous frame, output from the power spectrum calculating unit 23 (step S54) and adds the frequency index number to the memory (step S55). At step S56, the similarity calculating unit 34 determines if the current index is the final index and if not (step S56: NO), the similarity calculating unit 34 shifts frequency k (index number +1) (step S57) and returns to step S53. On the other hand, if the current index is the final index (step S56: YES), the similarity calculating unit 34 reads out the calculated average values of the power spectra and the index numbers from the memory, performs the above correlation calculation (step S58), and ends the processing.

According to the fourth embodiment, similarity can be prevented from being heightened between the frames in which only the background noise is present and the breathing state can be detected more accurately. Further, the detection of the breathing state can be processed using the general-purpose correlation equation.

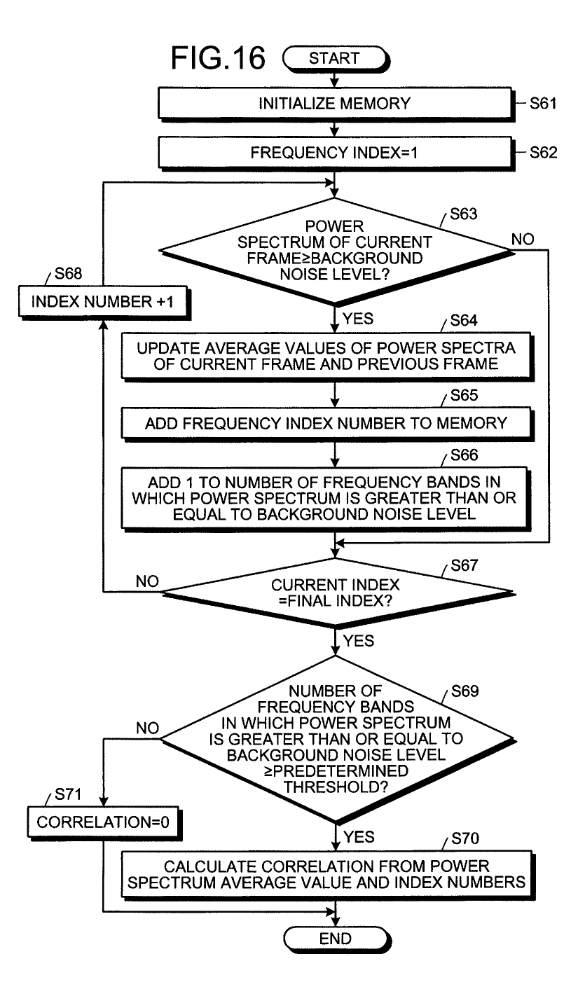

A fifth embodiment is a variation example of the fourth embodiment and represents the processing on the assumption that the background noise level is too great to detect the breathing state. FIG. 16 is a flowchart of the similarity calculating processing according to the fifth embodiment. The similarity calculating unit 34 first initializes the memory (step S61). The memory to be initialized is a memory to store the average value of the power spectrum of the current frame and the average value of the power spectrum of the previous frame (buffer 27 depicted in FIG. 11). The memory disposed in the background noise estimating unit 32 to hold the index of the frequency band in which the power spectrum is greater than or equal to the background noise level is also included among the memory to be initialized.

The similarity calculating unit 34 sets the index of frequency k to 1 (index=1) (step S62). The similarity calculating unit 34 determines if the power spectrum of the current frame is greater than or equal to the background noise level (step S63). If the power spectrum of the current frame is greater than or equal to the background noise level (step S63: YES), the similarity calculating unit 34 continues to perform the processing of step S64 and thereafter, but if the power spectrum of the current frame is less than the background noise level (step S63: NO), the flow goes to step S67.

At step S63, if the power spectrum of the current frame is greater than or equal to the background noise level (step S63: YES), the similarity calculating unit 34 updates the average values of the power spectra of the current frame and the previous frame, output from the power spectrum calculating unit 23 (step S64) and adds the frequency index number to the memory (step S65). The similarity calculating unit 34 then adds 1 to the number of the frequency bands in which the power spectrum is greater than or equal to the background noise level and stores the number to the memory (step S66).

At step S67, the similarity calculating unit 34 determines if the current index is the final index and if not (step 67: NO), the similarity calculating unit 34 shifts frequency k (index number +1) (step S68) and returns to step S63. On the other hand, if the current index is the final index (step S67: YES), the similarity calculating unit 34 reads out from the memory, the number of frequency bands in which the power spectrum is greater than or equal to the background noise level and determines if this number of frequency bands is greater than or equal to a predetermined threshold (step S69). If the number of the frequency bands in which the power spectrum is greater than or equal to the background noise level is greater than or equal to the threshold (step S69: YES), the similarity calculating unit 34 reads out the calculated power spectrum average values and the index numbers from the memory, performs the above correlation calculation (step S70), and ends the processing. On the other hand, if the number of frequency bands in which the power spectrum is greater than or equal to the background noise level is less than the threshold (step S69: NO), the similarity calculating unit 34 determines the correlation as 0 (non-existent) (step S71) and ends the processing without performing the correlation calculating processing.

The threshold is set as a value corresponding to the number of bands of frequency k in the power spectrum calculating unit 23 and if the number of bands of frequency k is 64 steps, for example, a value of 30 to 40, which is the value on the order of 60% of the number of steps, is set as the threshold. According to the fifth embodiment, when the background noise level is high, the breathing state is considered to be undetectable and the correlation calculation is not performed, thereby enabling environmental changes at the time of occurrence of the background noise to be coped with and achieving efficient processing.

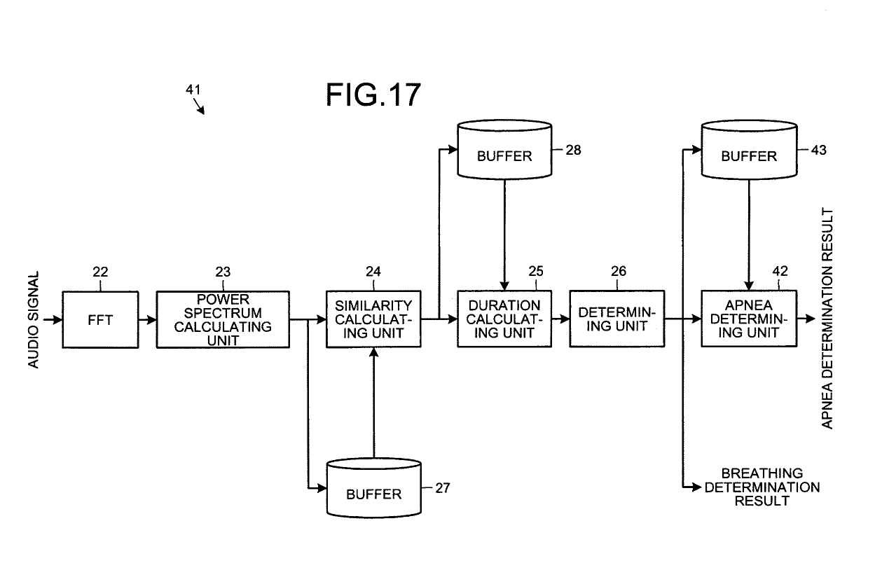

A sixth embodiment is configured to determine a state of apnea by adding an apnea determining unit to the configuration of the first embodiment. FIG. 17 is a block diagram of the sound processing apparatus according to the sixth embodiment. A sound processing apparatus 41 depicted in FIG. 17 has an apnea determining unit 42 disposed downstream from the determining unit 26 and determines a state of apnea upon receipt of output from the determining unit 26. Past results of the breathing determination by the determining unit 26 are sequentially stored to a buffer 43 and the apnea determining unit 42 determines the state of apnea, using current results of the breathing determination and the past results of the breathing determination stored in the buffer 43.

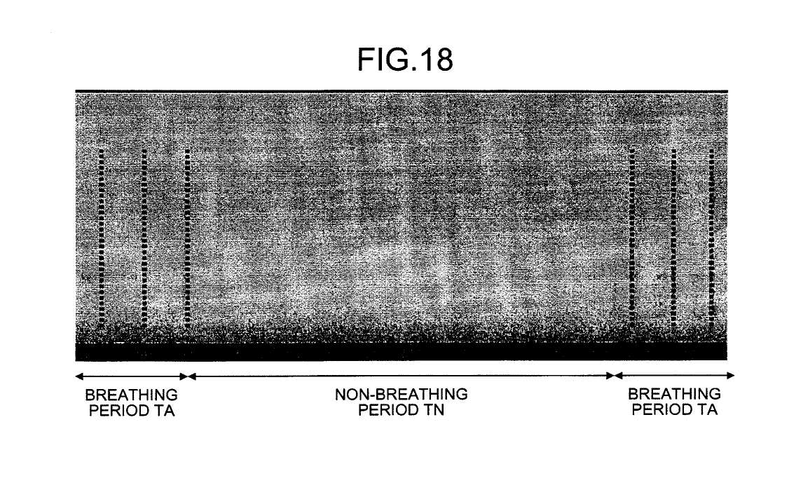

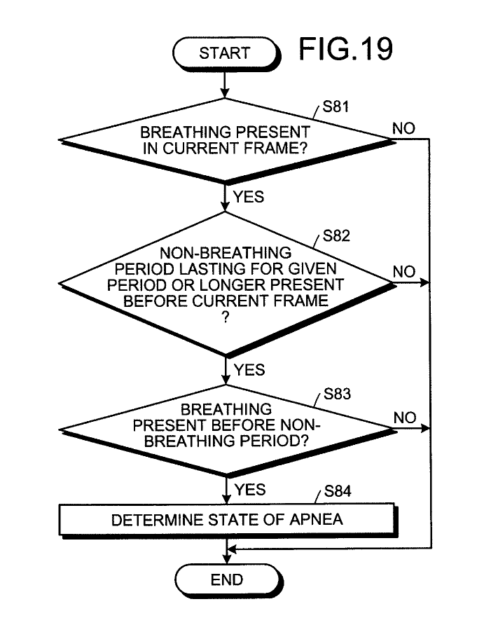

FIG. 18 is a diagram for describing a non-breathing state. As depicted in FIG. 18, even if a non-breathing state of a given period TN occurs, there is a breathing period TA before and after this non-breathing period TN. FIG. 19 is a flowchart of apnea determining processing of the sixth embodiment. The apnea determining unit 42 determines a state of apnea based on the state depicted in FIG. 18.

The apnea determining unit 42 first determines, based on the breathing determination results, if breathing is present in the current frame (step S81). If breathing is present in the current frame (step S81: YES), the apnea determining unit 42 reads out past results of the breathing determination from the buffer 43 and determines if there is a non-breathing period lasting for the given period (TN) or longer before the current frame (step S82). If there is a non-breathing period lasting for the given period (TN) or longer before the current frame (step S82: YES), the apnea determining unit 42 determines if breathing is present before the non-breathing period at step S82 (step S83). If breathing is present (step S83: YES), the apnea determining unit 42 determines a state of apnea (step S84) and ends the processing. On the other hand, if the determination results at step S81, step S82, or step S83 are NO, the apnea determining unit 42 ends the processing without performing the apnea determination.

According to the sixth embodiment, based on the manner of transition to a state of apnea, a state of apnea can be determined accurately by detecting the presence or absence of breathing from past data sequentially. While the breathing state may be undetectable depending on the direction or the condition of a microphone to acquire a sound signal, the above processing can ultimately determine that the sound signal indicates a state of apnea after detecting both the breathing state and the non-breathing state and thus, makes it possible to accurately determine a state of apnea even consequent to a change in the performance of the microphone, etc. The sound processing apparatus 41 or an external device may be designed to output an alarm upon receipt of determination results indicating a state of apnea, enabling further use in monitoring babies, young children, the elderly under nursing care, etc.

The sound processing apparatus described above can be configured using, for example, a cellular phone. The breathing state can be detected simply and accurately by executing a breathing detection program of the cellular phone at bedtime. Application is not limited to a cellular phone and the same breathing detection can be realized using mobile devices such a personal computer, a personal digital assistant (PDA), etc., having a built-in microphone. The microphone may be externally connected.

The breathing detection method described in the embodiments can be implemented by executing a prepared program on a computer such as a personal computer and a workstation. The breathing detecting processing described above can be performed by causing a CPU of the computer to execute the sound processing (breathing detection) program stored in a ROM, etc. At the time of such an execution, the CPU, using a RAM, etc., (not depicted) as a work area, implements the functions of the FFT to the determining unit. Sound (breathing during sleep) as the sound signal is detected by the microphone as a voltage value and the results of the breathing determination are output for display on a display unit. The buffers can be configured using a memory unit such as the RAM. The program is recorded on a computer-readable recording medium such as a hard disk, a flexible disk, a CD-ROM, an MO, and a DVD and is executed by being read out by the computer from the recording medium. The program may be a transmission medium that can be distributed by way of a network such as the Internet.

All examples and conditional language provided herein are intended for pedagogical purposes of aiding the reader in understanding the invention and the concepts contributed by the inventor to further the art, and are not to be construed as limitations to such specifically recited examples and conditions, nor does the organization of such examples in the specification relate to a showing of the superiority and inferiority of the invention. Although one or more embodiments of the present invention have been described in detail, it should be understood that the various changes, substitutions, and alterations could be made hereto without departing from the spirit and scope of the invention.

What is claimed is:

1. A sound processing apparatus comprising a processor configured to:
convert an input audio signal into a frequency domain signal;
calculate similarity of the current frequency domain signal and a previous frequency domain signal; and
determine a breathing state of a biological entity indicated by the audio signal, based on the similarity.

2. The sound processing apparatus according to claim 1, the processor further configured to:
calculate a power spectrum of each frequency of the frequency domain signal, using the current frequency domain signal, wherein
the processor calculates the similarity, using a current power spectrum and a previous power spectrum.

3. The sound processing apparatus according to claim 1, the processor further configured to
calculate a duration period of the audio signal, using the similarity, wherein
the processor determines the breathing state, based on the duration period.

4. The sound processing apparatus according to claim 2, wherein
the processor calculates for each frequency band, the similarity by comparing the current power spectrum and the previous power spectrum that is within a predetermined temporal range from the current power spectrum.

5. The sound processing apparatus according to claim 2, the processor further configured to
estimate a level of background noise in the audio signal, based on the power spectrum, wherein
the processor calculates the similarity, using only the frequency band in which the magnitude of the power spectrum is greater than the level of background noise.

6. The sound processing apparatus according to claim 2, wherein
the processor calculates a correlation of the current power spectrum calculated by the power spectrum calculating unit and the past power spectrum and uses the correlation as the similarity.

7. The sound processing apparatus according to claim 6, wherein
the processor calculates the correlation, using only the frequency band in which the magnitude of the power spectrum is greater than the level of background noise.

8. The sound processing apparatus according to claim 7, wherein
the processor determines the similarity as zero when the number of frequency bands in which the magnitude of the power spectrum is greater than the level of background noise, is less than or equal to a predetermined threshold.

9. The sound processing apparatus according to claim 3, wherein
the processor, using a current similarity and a previous similarity, determines the duration period as a period of the audio signal, in which the similarity is greater than or equal to a predetermined threshold.

10. A breathing detection method executed by a processor, the sound processing method comprising:
converting an input audio signal into a frequency domain signal;
calculating similarity of the current frequency domain signal and a previous frequency domain signal; and
determining a breathing state of a biological entity indicated by the audio signal, based on the similarity.
